(19) United States
(12) Patent Application Publication
(10) Pub. No.: US 2021/0261479 A1
(43) Pub. Date: Aug. 26, 2021

(54) METHOD AND APPARATUS FOR FORMING HYDROCARBONS

(71) Applicant: TEKNOLOGIAN TUTKIMUSKESKUS VTT OY, Espoo (FI)

(72) Inventors: Matti REINIKAINEN, Espoo (FI); Pekka SIMELL, Espoo (FI); Johanna KIHLMAN, Espoo (FI); Noora KAISALO, Espoo (FI)

(73) Assignee: TEKNOLOGIAN TUTKIMUSKESKUS VTT OY, Espoo (FI)

(21) Appl. No.: 16/973,806

(22) PCT Filed: Jun. 10, 2019
(86) PCT No.: PCT/FI2019/050447

§ 371 Date: Dec. 10, 2020

(30) Foreign Application Priority Data
Jun. 11, 2018 (FI) 20185531

(51) Int. Cl. C07C 1/12

(52) U.S. Cl. CPC C07C 1/12; C07C 2523/745; C07C 2523/75

(57) ABSTRACT

A method and an apparatus for forming hydrocarbons. A feed which includes at least carbon dioxide supplied to a reactor having two catalysts, which are a Fe-based catalyst and a Co-based catalyst, and the catalysts are arranged inside the same reactor, and hydrogen is fed into the reactor. The feed is arranged to flow through the reactor and arranged to contact with the hydrogen and the catalysts in the reactor, and the feed is treated by two reaction steps wherein carbon monoxide is formed from the carbon dioxide and hydrogen and wherein hydrocarbons are formed from the carbon monoxide and hydrogen in the reactor.

### FIELD

The application relates to a method defined in claim 1 and an apparatus defined in claim 10 for forming hydrocarbons. Further, the application relates to a use of the method defined in claim 15.

### BACKGROUND

Known from the prior art is to produce hydrocarbons by a Fischer-Tropsch synthesis. The Fischer-Tropsch synthesis requires a mixture of H₂ and CO as feed. There are commercial processes available for production of fuels, olefins or oxygenates.

Further, it is known from the prior art that carbon dioxide may be converted to carbon monoxide by RWGS (reverse water gas shift) reaction. However, RWGS reaction is a highly endothermic reaction requiring high reaction temperature.

### OBJECTIVE

The objective is to disclose a new type method and apparatus for producing hydrocarbons from carbon dioxide. Further, the objective is to disclose a new type method and apparatus for treating carbon dioxide streams. Further, the objective is to improve a Fischer-Tropsch synthesis.

### SUMMARY

The method and apparatus and use are characterized by what are presented in the claims.

### BRIEF DESCRIPTION OF THE DRAWINGS

The accompanying drawings, which are included to provide a further understanding of the invention and constitutes a part of this specification, illustrate some embodiments of the invention and together with the description help to explain the principles of the invention. In the drawings:

FIG. 1 is a flow chart illustration of a process according to one embodiment,

FIG. 2 is a flow chart illustration of a process according to another embodiment,

FIG. 3 is a flow chart illustration of a process according to another embodiment,

FIG. 4 is a flow chart illustration of a process according to another embodiment, and

FIG. 5 shows test results.

### DETAILED DESCRIPTION

In a method for forming hydrocarbons, a feed (1) which comprises at least carbon dioxide is supplied to a reactor (2) comprising two catalysts, which are a Fe-based catalyst (6) and a Co-based catalyst (7), and said catalysts are arranged inside the same reactor, hydrogen (4) is fed into the reactor, and the feed is arranged to flow through the reactor and arranged to contact with the hydrogen and the catalysts in the reactor, and the feed is treated by means of two reaction steps wherein carbon monoxide is formed from the carbon dioxide and hydrogen and wherein hydrocarbons (3) are formed from the carbon monoxide and hydrogen in the reactor. In the method carbon dioxide is converted with hydrogen to hydrocarbons. In one embodiment, olefin-rich hydrocarbons are formed, and paraffin-rich hydrocarbons are formed from the olefin-rich hydrocarbons in the reactor.

An apparatus for forming hydrocarbons comprises at least one reactor (2) to which a feed (1) comprising at least carbon dioxide is supplied, two catalysts, which are a Fe-based catalyst (6) and a Co-based catalyst (7), and said catalysts are arranged inside the same reactor, and a feeding device for feeding hydrogen (4) into the reactor. In the reactor the feed is arranged to flow through the reactor and arranged to contact with the hydrogen and the catalysts for treating the feed by means of two reaction steps in order to form carbon monoxide from the carbon dioxide and hydrogen and to form hydrocarbons (3) from the carbon monoxide and hydrogen.

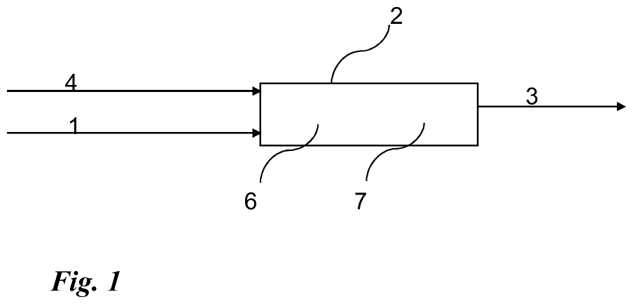

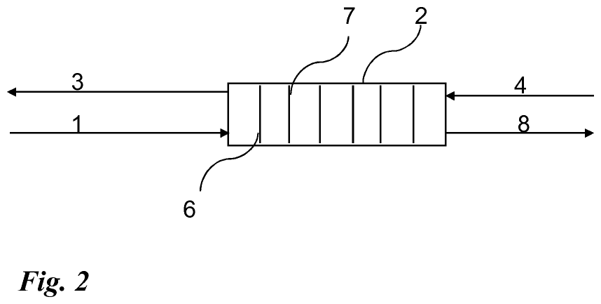

One embodiment of the method and the apparatus is shown in FIG. 1. Other embodiments of the method and the apparatus are shown in FIGS. 2, 3 and 4.

Preferably, the feed (1) of the reactor (2) is in gaseous form. In this context, the feed means any feed which comprises at least carbon dioxide. Further, the feed can comprise other components, e.g. carbon monoxide. The feed can contain one or more components. In one embodiment, the feed consists of mainly carbon dioxide. In one embodiment, the feed comprises at least carbon dioxide and at least carbon monoxide. A ratio of carbon dioxide and carbon monoxide can vary in the feed. The feed can comprise also other components, e.g. inert components, hydrocarbons, water, hydrogen and/or other components. In one embodiment, the feed comprises at least carbon dioxide and at least one hydrocarbon. In one embodiment, the feed may comprise hydrogen. The feed can be supplied to a catalyst bed of the reactor. In one embodiment, the feed is treated before the supply into the reactor. In one embodiment, the feed is a flow from a gas recirculation system. In one embodiment, the feed is flow from a flue gas system or from burning of carbonaceous matter. In one embodiment, the feed is formed from air or air of ventilation system. In one embodiment, the feed is formed from carbon dioxide based flow of the industrial process.

Preferably, the both catalysts (6,7) are arranged into the same reactor (2), such as into the single reactor or into one reactor. Also the both reaction steps are carried out in the same reactor.

In one embodiment, the Fe-based catalyst (6) is Fe/Al₂O₃ catalyst or other suitable Fe-based catalyst. In one embodiment, the Co-based catalyst (7) is Co/Al₂O₃ catalyst or other suitable Co-based catalyst.

In one embodiment the catalyst, such as the Fe-based (6) or Co-based (7) catalyst, is arranged as a coating on a desired substrate, e.g. carrier surface, to form a catalyst surface. In one embodiment the substrate can be a surface of plate, pipe, tube or the like. In one embodiment the catalyst is arranged as the coating onto a metal substrate, in which metal can be any metal, e.g. steel, aluminum, other metal or their combination. In one embodiment, the catalyst is arranged as the coating onto a ceramic substrate. In one embodiment, the catalyst is arranged as a coating on a substrate, e.g. as a washcoating, onto a metal surface, such as metal monolith, or ceramic surface, such as ceramic monolith.

In one embodiment, the reactor (2) comprises at least one catalyst arrangement which contains both Fe-based catalyst (6) and Co-based catalyst (7). In one embodiment, the reactor comprises at least one first catalyst zone which contains Fe-based catalyst or Co-based catalyst and at least one second catalyst zone which contains Co-based catalyst or Fe-based catalyst. In one embodiment, the reactor comprises at least one first catalyst zone which contains Fe-based catalyst and at least one second catalyst zone which contains Co-based catalyst. In one embodiment, the reactor comprises at least one first catalyst surface which contains Fe-based catalyst and at least one second catalyst surface which contains Co-based catalyst. In one embodiment, the Fe-based catalyst and Co-based catalyst are arranged in the reactor such that firstly there is the Fe-based catalyst, e.g. catalyst zone or surface containing Fe-based catalyst, and secondly there is the Co-based catalyst, e.g. catalyst zone or surface containing Co-based catalyst, in the direction of the feed flow in the reactor. In one embodiment, the Fe-based and Co-based catalysts are arranged to different parts, zones or areas, preferably to desired parts, zones or areas, inside the reactor.

In one embodiment, the reactor (2) is a heat exchanger type reactor in which heat is transferred from an exothermic reaction to an endothermic reaction.

In one embodiment, the reactor (2) is a plate heat exchanger type reactor in which a part of the plates are catalytically coated with the Fe-based catalyst (6) layer and a part of the plates are catalytically coated with the Co-based catalyst (7) layer. In one embodiment, the reactor is a plate heat exchanger type reactor in which each plate is coated partly with the Fe-based catalyst (6) layer and partly with the Co-based catalyst (7) layer. In one embodiment, the plates with the Fe-based catalyst (6) and the plates with the Co-based catalyst (7) are placed consecutively in the heat exchanger type reactor. In one embodiment, the number and order of the plates are chosen so that the reaction heat between the exothermic reaction and endothermic reaction can be divided in optimal way. In one embodiment, the catalytically coated plates are stacked so that temperature is higher on the Fe-based plate or zone and is lower on the Co-based plate or zone for ensuring an ideal product distribution. Preferably, the feed is supplied to the desired part, e.g. to a desired interspace of the plates, in the reactor.

In one embodiment, the reactor (2) is a tube reactor or tubular reactor, e.g. tube heat exchanger type reactor. In one embodiment, the reactor is a tubular reactor which is a microchannel reactor. In one embodiment, the tube reactor, e.g. tube heat exchanger type reactor, comprises two tubes so that the first tube is inside the second tube and the first catalyst, such as Fe-based catalyst (6) or Co-based catalyst (7), is on an outer surface of the first tube and the second catalyst, such as Co-based catalyst (7) or Fe-based catalyst (6), is on an inner surface of the second tube. In one embodiment, the Fe-based catalyst is arranged on an outer surface of the first tube and the Co-based catalyst is arranged on an inner surface of the second tube. In one embodiment, the Co-based catalyst is arranged on an outer surface of the first tube and the Fe-based catalyst is arranged on an inner surface of the second tube. Preferably, a heat-transfer agent flows inside the first tube. In one embodiment, the tube reactor comprises one tube, and the first catalyst, such as Fe-based catalyst or Co-based catalyst, is arranged as inserts inside the tube and the second catalyst, such as Co-based catalyst or Fe-based catalyst, is on an inner surface of the tube. In one embodiment, the Fe-based catalyst is arranged as inserts inside the tube and the Co-based catalyst is arranged on an inner surface of the tube. In one embodiment, the Co-based catalyst is arranged as inserts inside the tube and the Fe-based catalyst is arranged on an inner surface of the tube. In one embodiment, the first catalyst, such as Fe-based catalyst or Co-based catalyst, is arranged on the inner tube and the second catalyst, such as Co-based catalyst or Fe-based catalyst, is arranged on the inner surface of the reactor casing in the tube heat exchanger type reactor. In one embodiment, a heat-transfer agent flows inside the inner tube.

In one embodiment, the hydrogen (4) is used as a reactant in the reactor (2). In one embodiment, the hydrogen is supplied into the reactor by means of one feed inlet. In one embodiment, the hydrogen is supplied into the reactor by means of at least two feed inlets. Preferably, the hydrogen is supplied to a desired part of the reactor. In one embodiment, the hydrogen is supplied from the opposite direction than the feed (1) into the reactor. In one embodiment, the hydrogen transfers heat from an exothermic reaction, such as from FT-reaction, to an endothermic reaction, such as to RWGS reaction, especially in heat exchanger type reactors.

In one embodiment, two reaction steps which are a reverse water gas shift reaction (RWGS) and a Fischer-Tropsch reaction (FT) steps are carried out in the reactor (2). The reverse water gas shift (RWGS) reaction is an endothermic reaction. Preferably, carbon dioxide is converted to at least carbon monoxide, i.e. carbon monoxide is formed from the carbon dioxide and hydrogen, in the reverse water gas shift reaction step. Further, olefin-rich hydrocarbons, such as light hydrocarbons, may be formed in the reverse water gas shift reaction step. Alternatively, the olefin-rich hydrocarbons, such as light hydrocarbons, may be formed in the reverse water gas shift reaction and Fischer-Tropsch reaction steps. The Fischer-Tropsch (FT) reaction is an exothermic reaction. Preferably, paraffin-rich hydrocarbons, which can be considered as heavy hydrocarbons, are formed from the carbon monoxide, hydrogen and olefin-rich hydrocarbons in the Fischer-Tropsch (FT) reaction step, and preferably olefinic hydrocarbons are hydrogenated by the Co-based catalyst into paraffin hydrocarbons. The heat from the exothermic reaction is used in the endothermic reaction. Preferably, the FT reaction brings the necessary heat for the reaction in which carbon dioxide is converted to carbon monoxide. Further, the products from the first reaction, e.g. from RWGS reaction, are utilized as start compounds in the second reaction, e.g. in FT reaction. In the reactor the reactions are carried out in series, sequentially, consecutively, in turn, in a random order, in a predetermined order, or in the order according to their combination.

In one embodiment, the first reaction is a RWGS reaction and the second reaction is a FT reaction in the reactor. In one embodiment, the first reaction is carried out on the Fe-based catalyst (6) surface. In one embodiment, the second reaction is carried out on the Co-based catalyst (7) surface. In one embodiment, the first reaction is carried out on the Fe-based catalyst surface and the second reaction is carried out on the Co-based catalyst surface. In one embodiment, the first reaction is carried out on the Fe-based catalyst surface and the second reaction is carried out on the both Fe-based and Co-based catalyst surfaces.

Preferably, the high activity reaction with the Co-catalyst (7) consumes carbon monoxide for driving the equilibrium of the reaction with the Fe-catalyst (6) to the desired direction. In one embodiment, the FT reaction consumes carbon monoxide for pushing the equilibrium of the RWGS reaction to the desired direction.

Preferably, the invention is based on the combination of the RWGS reaction and the FT reaction. In one embodiment, the invention is based on a combined RWGS- and FT-reactor. In the combined reactor, heat integration between the exothermic FT reaction and endothermic RWGS reaction can be achieved.

In one embodiment, temperature of the feed (1) can be varied or adjusted. In one embodiment, the temperature of the feed is adjusted on grounds of a reactor construction, a desired reaction temperature or their combination. In one embodiment, the feed can be used as a heat transfer material simultaneously when the feed is supplied to the reactor (2). In one embodiment, the feed is heated before supplying into the reactor (2), especially if the reactor is not a heat exchanger type reactor.

In one embodiment, the treatment temperature is 100-500° C. in the reactor (2). In one embodiment, the treatment temperature is 150-370° C. in the reactor, and in one embodiment 170-350° C. in the reactor. In one embodiment, the RWGS reaction is carried out at temperature which is 190-400° C., preferably 200-350° C., in the reactor. In one embodiment, the FT reaction is carried out at temperature which is 130-270° C., in one embodiment 150-250° C., preferably 190-220° C., in the reactor. Preferably, reaction heat is utilized in the endothermic reactions, such as RWGS reactions, which take place in the same reactor. In one embodiment, the reactions are started by heating the feed, e.g. by means of an external heat device, before the supply of the feed into the reactor.

In one embodiment, pressure is 1-50 bar, preferably 5-30 bar in the reactor (2).

In one embodiment, a product is formed from the hydrocarbons (3) formed in the reactor (2). The hydrobarbons (3) comprise a mixture of different hydrocarbons, e.g. C5-C30 hydrocarbons. In this context, the product means any product comprising at least the hydrocarbons (3). The product comprises one or more product components, e.g. different hydrocarbons, carbon monoxide, hydrogen and/or other components. In one embodiment, the product is a mixture of hydrocarbons. In one embodiment, the product comprises at least hydrocarbons, preferably C5-C30 hydrocarbons. In one embodiment, the product comprises at least gasoline range hydrocarbons, such as C5-C12 hydrocarbons. In one embodiment, the product may comprise also other organic compounds. In one embodiment, non-condensable components can be discharged or separated from the product after the reactor (2). In one embodiment, the product is in form of liquid.

In one embodiment, H₂/CO ratio can be adjusted by means of an amount of components of the feed (1) to the reactor (2).

In one embodiment, the hydrocarbons (3) can be post-treated after the reactor (2). In one embodiment, the hydrocarbons can be supplied to a desired treatment process, e.g. for refining hydrocarbons.

In one embodiment, the method comprises more than one treatment stage. In one embodiment, the apparatus comprises more than one reactor (2). In one embodiment, the method comprises one treatment stage. In one embodiment, the apparatus comprises one reactor.

In one embodiment, at least two reactors are arranged in parallel. In one embodiment, at least two reactors are arranged sequentially.

In one embodiment, the apparatus comprises at least one outlet for discharging the hydrocarbons (3) out from the reactor (2).

In one embodiment, the apparatus comprises at least one feed inlet for supplying the feed (1) into the reactor (2).

The feed inlet may be any suitable inlet known per se, e.g. pipe, port or the like. The hydrocarbon outlet may be any suitable outlet known per se, e.g. pipe, outlet port or the like.

Preferably, the apparatus comprises at least one feeding device. In this context, the feeding device can be any feeding device, equipment or other suitable device. In one embodiment, the feeding device is selected from the group comprising pump, compressor, tube, pipe, other suitable feeding device and their combinations.

In one embodiment, the method is based on a continuous process. In one embodiment, the apparatus is a continuous apparatus. In one embodiment, the method is based on a batch process. In one embodiment, the apparatus is a batch apparatus.

In one embodiment, the apparatus and the method is used and utilized in a production of hydrocarbons, Fischer-Tropsch (FT) process, treatment of carbon dioxide, carbon dioxide capture process, reduction of carbon dioxide emissions, manufacturing of fuels, methanation process, production of methanol, or their combinations.

Thanks to the invention hydrocarbons can be produced from carbon dioxide based feed easily and effectively. Temperature can be kept low in the reactor. The Fischer-Tropsch reaction step brings the necessary heat for the reaction in which carbon dioxide is converted to carbon monoxide. A separate heating device or cooling device is not needed in the reactor. Typically, high exothermicity of the Fischer-Tropsch reaction subjects to mass-transfer limitations and unideal temperature profile during the reaction. On the other hand, the RWGS reaction is limited by thermodynamic equilibrium requiring high temperatures. Now, thanks to the invention the heat formed in the Fischer-Tropsch reaction can be utilized in the RWGS reaction. At the same time, heat transfer, mass transfer and equilibrium restrictions can be overcome.

The method and apparatus offers a possibility to form hydrocarbon products with good properties easily, and energy- and cost-effectively. The present invention provides an industrially applicable, simple and affordable way to treat carbon dioxide, and further simultaneously to produce hydrocarbons. The method and apparatus are easy and simple to realize in connection with production processes.

### EXAMPLES

### Example 1

FIG. 1 presents the method and also the apparatus for producing hydrocarbons from carbon dioxide (CO₂).

A feed (1) which comprises at least carbon dioxide is supplied to a reactor (2) comprising two catalysts, which are a Fe-based catalyst (6) and a Co-based catalyst (7), and said catalysts are arranged inside the same reactor. Hydrogen (4) is fed into the reactor (2). The feed is arranged to flow through the reactor and arranged to contact with the hydrogen (4) and the both catalysts (6,7) in the reactor. The feed is treated by means of two reaction steps in which carbon monoxide is formed from the carbon dioxide and hydrogen by means of Fe-based catalyst and in which hydrocarbons (3) are formed from the carbon monoxide and hydrogen by means of Fe-based and Co-based catalyst. Olefinic hydrocarbons may be formed firstly, and paraffin hydrocarbons may be formed from the olefinic hydrocarbons. In the method, carbon dioxide is converted with hydrogen to hydrocarbons.

### Example 2

FIG. 2 presents the method and also the apparatus for producing hydrocarbons from carbon dioxide (CO₂).

A feed (1) which comprises at least carbon dioxide is supplied to a reactor (2) comprising two catalysts, which are a Fe-based catalyst (6) and a Co-based catalyst (7), and said catalysts are arranged inside the same reactor. Hydrogen (4) is fed into the reactor (2). The feed is arranged to flow through the reactor and arranged to contact with the hydrogen (4) and the catalysts (6,7) in the reactor. The feed is treated by means of two reaction steps in which carbon monoxide is formed from the carbon dioxide and hydrogen and in which hydrocarbons (3) are formed from the carbon monoxide and hydrogen in the reactor.

The reactor (2) is a plate heat exchanger type reactor, and simultaneously the reactor is a combined RWGS- and FT-reactor. In the reactor a part of the plates are catalytically coated with the Fe-based catalyst layer and a part of the plates are catalytically coated with the Co-based catalyst layer. In the reactor of FIG. 2, each plate is coated partly with the Fe-based catalyst layer and partly with the Co-based catalyst layer. Alternatively, the reactor comprises plates which are coated with Fe-based catalyst and plates which are coated with Co-based catalyst, and the plates with the Fe-based catalyst and the plates with the Co-based catalyst are placed consecutively in the reactor. Preferably, the catalytically coated plates are stacked so that the temperature is higher on the Fe-based zone of the plate and the temperature is lower on the Co-based zone of the plate for ensuring an ideal product distribution. The hydrogen (4) transfers heat in the reactor. The feed (1) is supplied to the reactor so that the gaseous feed first reacts on the Fe-based zone of the plates and formed carbon monoxide reacts further to hydrocarbons both on the Fe- and Co-based zones of the plates, and the hydrogen (4) is supplied from the opposite direction to the reactor. First the carbon dioxide is converted to the carbon monoxide, and further olefin-rich hydrocarbons are formed mainly on the Fe-based zone of the plates. After that paraffin-rich hydrocarbons are formed from the olefin-rich hydrocarbons on the Co-based zone of the plates. Preferably, the feed is supplied to the reactor such that firstly a reaction with the Fe-based catalyst can be carried out and secondly reactions with the Fe- and Co-based catalysts can be carried out in direction of the feed flow in the reactor.

The method comprises two reaction steps which are an endothermic RWGS-reaction (reverse water gas shift reaction) and an exothermic FT-reaction (Fischer-Tropsch reaction). In these reactions, carbon monoxide is formed from carbon dioxide and hydrogen, and hydrocarbons are formed from the carbon monoxide and hydrogen. The FT-reaction consumes carbon monoxide for pushing the equilibrium of the RWGS-reaction to the right direction. The olefinic hydrocarbons formed on the Fe-based catalyst zone of the plates reacts to higher paraffins on the Co-based catalyst zone of the plates. The heat from the exothermic FT-reaction is transferred by means of the hydrogen to the endothermic RWGS-reaction.

A product comprising the formed hydrocarbons (3) is discharged from the reactor (2). Further, a gas stream (5) may be discharged from the reactor.

### Example 3

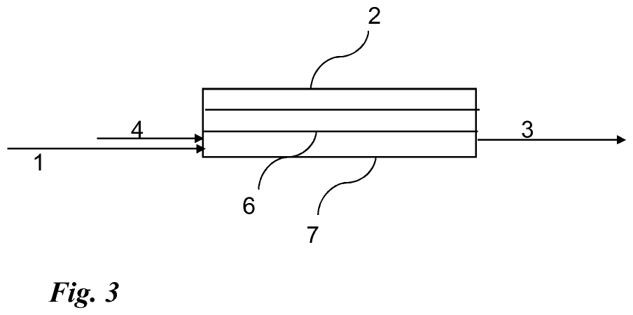

FIG. 3 presents the method and also the apparatus for producing hydrocarbons from carbon dioxide (CO₂).

A feed (1) which comprises at least carbon dioxide is supplied to a reactor (2) comprising two catalysts, which are a Fe-based catalyst (6) and a Co-based catalyst (7), and said catalysts are arranged inside the same reactor. Hydrogen (4) is fed into the reactor (2). The feed is arranged to flow through the reactor and arranged to contact with the hydrogen (4) and the catalysts (6,7) in the reactor, and the feed is treated by means of two reaction steps in which carbon monoxide is formed from the carbon dioxide and hydrogen and in which hydrocarbons (3) are formed from the carbon monoxide and hydrogen in the reactor. In the method carbon dioxide is converted with hydrogen to hydrocarbons by means of two reaction steps.

The reactor (2) is a tube heat exchanger type reactor. This tube reactor comprises two tubes so that the first tube is inside the second tube. The first catalyst, i.e. the Fe-based catalyst, is arranged on an outer surface of the first tube and the second catalyst, the Co-based catalyst, is arranged on an inner surface of the second tube. The feed (1) and hydrogen (4) are supplied into between the first and second tubes, and a heat-transfer agent flows inside the first tube.

The two reaction steps, in which carbon monoxide is formed from carbon dioxide and hydrogen by the Fe-based catalyst and hydrocarbons are formed from the carbon monoxide and hydrogen by Fe-based and Co-based catalysts, is carried out inside the outer tube.

A product comprising the formed hydrocarbons (3) is discharged from the reactor (2).

### Example 4

In this example the catalysts were formed and they were packed into a vertical reactor which corresponds to the reactor according to FIG. 1.

A cobalt and an iron catalyst were prepared by using known methods. A cobalt catalyst (LSC-41) was prepared by impregnation of a water solution of Co(NO₃)₂x6H₂O on a Puralox SCFa-200-alumina which had been modified with tetraethoxysilane. The cobalt content of the ready catalyst was about 25 w-%. An iron catalyst (LSC-63) was prepared by impregnation of water solution of Fe (NO₃)₃x9H₂O on Puralox SCFa-200-alumina. The iron content of the ready catalyst was about 9 w-%.

The iron based catalyst (1.0 g of LSC-63) and the cobalt based catalyst (1.0 g of LSC-41) were packed consecutively in a vertical reactor tube so that the feed flows first through the iron containing catalyst and immediately after that the cobalt containing catalyst. The reactor tube was placed in an oven with two separately controlled heating zones.

### Example 5

The reactor set up in example 4 was used in this reaction test. The catalysts were activated for h with flowing hydrogen at 400° C., flow rate 0.1 l/min (STP) and atmospheric pressure.

After that, the temperatures were set to: 340° C. (upper part of the oven/Fe-catalyst) and 190° C. (lower part of the oven/Co-catalyst).

The feed was changed from hydrogen to a gas mixture comprising H₂ 71.25 vol-%, CO₂ 23.75 vol-%, N₂ 5.00 vol-% and pressurised to 20 barg. The flow rate was adjusted to 0.1 l/min (STP). The effluent was analysed by using an online gas-chromatograph.

After about 30 hours on stream the reaction had reached steady operation. The measured CO₂ conversion was about 45% and the chain growth probability alpha was 0.74 making a product rich in gasoline range hydrocarbons.

### Example 6

The invention was tested by using a system consisting of two nested metal tubes coated with catalytically active cobalt and iron layers.

Co-catalyst LSC-41 and Fe-catalyst LSC-63 from example 4 were used to make two slurries. The inner surface of the outer tube (Inconel 660, 18 mm od×2 mm) was coated with the Fe-containing slurry while the outer layer of the inner tube was coated with a Co-slurry. The coatings were done using known methods.

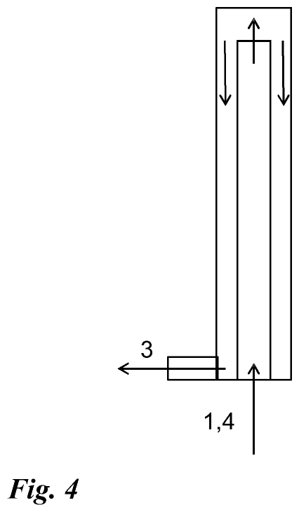

The principle and connection of the nested tubes are depicted in FIG. 4.

### Example 7

The reactor constructed in example 6 was used in this reaction test. The catalysts were activated for 18 h with flowing hydrogen at 400° C., flow rate 0.1 l/min (STP) and atmospheric pressure.

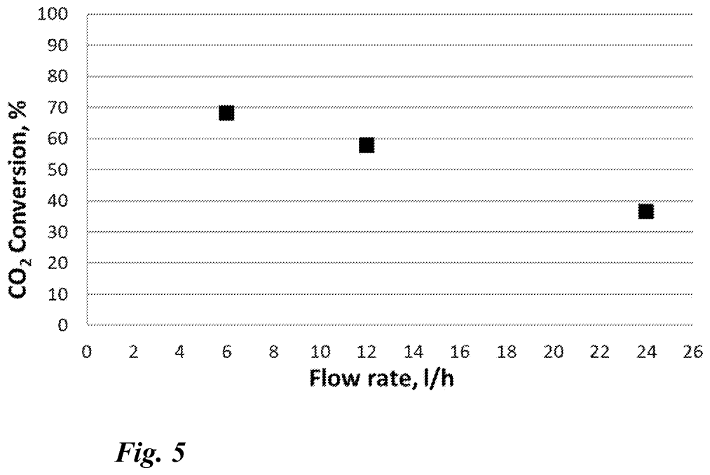

The temperature of the oven was set to 250° C. and the feed (1,4) was changed to a gas mixture comprising H₂ 71.25 vol-%, CO₂ 23.75 vol-%, N₂ 5.00 vol-%. The pressure was varied from 5 to 20 barg, the flow rate from 6 to 24 l/h and the oven setpoint from 250 to 400° C. The effluent, i.e. formed hydrocarbons (3), was analysed by using an online gas chromatograph. Mainly saturated hydrocarbons were formed as reaction product. An illustrative example of the CO₂ conversion is given in FIG. 5.

The feeding and outlet devices and recovering equipments of the process used in these examples are known per se in the art, and therefore they are not described in any more detail in this context.

The method and apparatus are suitable in different embodiments for treating carbon dioxide and for forming hydrocarbons from different kinds of feeds.

The invention is not limited merely to the examples referred to above; instead many variations are possible within the scope of the inventive idea defined by the claims.

1. A method for forming hydrocarbons, wherein
a feed which comprises at least carbon di-oxide is supplied to a reactor comprising two catalysts, which are a Fe-based catalyst and a Co-based catalyst, and said catalysts are arranged inside the same reactor,
hydrogen is fed into the reactor, and
the feed is arranged to flow through the reactor and arranged to contact with the hydrogen and the catalysts in the reactor, and the feed is treated by two reaction steps wherein carbon monoxide is formed from the carbon dioxide and hydrogen and wherein hydrocarbons are formed from the carbon monoxide and the hydrogen in the reactor.

2. The method according to claim 1, wherein a heat exchanger type reactor is used as the reactor in which heat is transferred from an exothermic reaction to an endothermic reaction.

3. The method according to claim 1, wherein the heat is transferred from the exothermic reaction to the endothermic reaction by the hydrogen.

4. The method according to claim 1, wherein the two reaction steps which are a reverse water gas shift reaction (RWGS) and a Fischer-Tropsch reaction (FT) steps are carried out in the reactor.

5. The method according to claim 1, wherein the first reaction which is the reverse water gas shift reaction is carried out on the Fe-based catalyst surface and the second reaction which is the Fischer-Tropsch reaction is carried out on the both Fe-based and Co-based catalyst surfaces.

6. The method according to claim 1, wherein the treatment temperature is 100-500° C. in the reactor.

7. The method according to claim 1, wherein the reactor comprises at least one first catalyst zone which contains Fe-based catalyst and at least one second catalyst zone which contains Co-based catalyst.

8. The method according to claim 1, wherein the catalyst is arranged as a coating on a desired substrate to form a catalyst surface.

9. The method according to claim 1, wherein the hydrocarbons comprise C5-C30 hydrocarbons.

10. An apparatus for forming hydrocarbons, wherein the apparatus comprises
at least one reactor to which a feed comprising at least carbon dioxide is supplied,
two catalysts, which are a Fe-based catalyst and a Co-based catalyst, and which are arranged inside the same reactor, and
a feeding device for feeding hydrogen into the reactor, and
in the reactor the feed is arranged to flow through the reactor and arranged to contact with the hydrogen and the catalysts for treating the feed by two reaction steps in order to form carbon monoxide from the carbon dioxide and hydrogen and to form hydrocarbons from the carbon monoxide and hydrogen.

11. The apparatus according to claim 10, wherein the reactor is a plate heat exchanger type reactor in which a part of the plates are catalytically coated with the Fe-based catalyst layer and a part of the plates are catalytically coated with the Co-based catalyst layer.

12. The apparatus according to claim 10, wherein the reactor is a plate heat exchanger type reactor in which each plate is coated partly with the Fe-based catalyst layer and partly with the Co-based catalyst layer.

13. The apparatus according to claim 10, wherein the apparatus comprises a tube reactor, and the tube re-actor comprises two tubes so that the first tube is inside the second tube and the first catalyst, which is Fe-based catalyst or Co-based catalyst, is arranged on an outer surface of the first tube and the second catalyst, which is Co-based catalyst or Fe-based catalyst, is on an inner surface of the second tube.

14. The apparatus according to claim 10, wherein the apparatus comprises a tube reactor, and the tube re-actor comprises one tube, and the first catalyst, which is Fe-based catalyst or Co-based catalyst, is arranged as inserts inside the tube and the second catalyst, which is Co-based catalyst or Fe-based catalyst, is on an inner surface of the tube.

15. The method according to claim 1, wherein the method is used in a production of hydrocarbons, Fischer-Tropsch (FT) process, treatment of carbon dioxide, carbon dioxide capture process, re-duction of carbon dioxide emissions, manufacturing of fuels, methanation process, production of methanol, or their combinations.
